(19) United States
(12) Patent Application Publication
(10) Pub. No.: US 2025/0177671 A1
(43) Pub. Date: Jun. 5, 2025

(54) INTUBATION MODE AND INTEGRATION WITH PATIENT ELECTRONIC MEDICAL RECORD

(71) Applicant: Covidien LP, Mansfield, MA (US)

(72) Inventors: Bo Chen, Louisville, CO (US); Derek S. Tata, Longmont, CO (US); Alexandra Hause, Denver, CO (US); Peter Douglas Colin Inglis, Boulder, CO (US); Paul S. Addison, Scotland (GB)

(73) Assignee: Covidien LP, Mansfield, MA (US)

(21) Appl. No.: 18/943,482

(22) Filed: Nov. 11, 2024

Related U.S. Application Data
(60) Provisional application No. 63/605,359, filed on Dec. 1, 2023.
(60) Provisional application No. 63/649,074, filed on May 17, 2024.

(51) Int. Cl. A61M 16/00; A61M 16/01; A61M 16/04; G06V 20/40; G16H 10/60

(52) U.S. Cl. CPC A61M 16/024; A61M 16/01; A61M 16/0488; G06V 20/41; G06V 20/46; G16H 10/60; A61M 2205/3306; A61M 2205/502; A61M 2230/432; G06V 2201/034

(57) ABSTRACT

The disclosed technology relates to an intubation mode and automatic data transfer to an electronic medical record for a patient. In an example the technology relates to an anesthesia machine that performs operations including receive sensor data from one or more connected sensors; display, on the display, patient parameters based on the received sensor data, wherein the patient parameters are displayed in a first display configuration; receive video data from a video laryngoscope; receive an indication to enter an intubation mode; in response to the indication, display, on the display, the video data and the patient parameters in a second display configuration; receive an indication to exit the intubation mode; and in response to the indication to exit the intubation mode, remove the video data from the display and display the patient parameters in the first display configuration.

### CROSS-REFERENCE TO RELATED APPLICATIONS

This application claims the benefit of U.S. Provisional Application No. 63/605,359 filed Dec. 1, 2023, and U.S. Provisional Application No. 63/649,074 filed May 17, 2024, entitled Intubation Mode and Integration with Patient Electronic Medical Record,” which is incorporated herein by reference its entirety.

### BACKGROUND

Video laryngoscopes are commonly used to perform intubations on patients who require breathing assistance. During an intubation, the video laryngoscope may be used to manipulate the anatomy of the larynx and associated structures of a patient's airway, in order to obtain a view sufficient for insertion of a breathing tube (e.g., an endotracheal tube) into the trachea. Intubation procedures often happen in combination with providing anesthesia to a patient to anesthetize the patient for a surgical procedure. As such, video laryngoscopes are frequently used in the presence of an anesthesia machine.

It is with respect to this general technical environment that aspects of the present technology disclosed herein have been contemplated. Furthermore, although a general environment is discussed, it shoul d be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

In an aspect, the technology relates to an anesthesia machine comprising a display; at least one processor; and memory storing instructions that, when executed by the at least one processor, cause the anesthesia machine to perform operations. The operations include receive sensor data from one or more connected sensors; display, on the display, patient parameters based on the received sensor data, wherein the patient parameters are displayed in a first display configuration; receive video data from a video laryngoscope; receive an indication to enter an intubation mode; in response to the indication, display, on the display, the video data and the patient parameters in a second display configuration; receive an indication to exit the intubation mode; and in response to the indication to exit the intubation mode, remove the video data from the display and display the patient parameters in the first display configuration.

In an example, the operations further comprise in response to the indication to enter the intubation mode, starting an intubation timer; and in response to the indication to exit the intubation mode, stopping the intubation timer. In a further example, the operations further comprise concurrently displaying the intubation timer with the video data and the patient parameters in the second display configuration. In another example, the operations further comprise, in response to the indication to exit the intubation mode, transmitting, to a patient terminal for inclusion in an electronic medical record, the video data receiving during the intubation mode and the patient parameters generated during the intubation mode. In still another example, the indication to enter the intubation mode is based on an absence of a capnography signal from a capnography sensor. In still a further another example, the indication to exit the intubation mode is based on detecting a capnography signal from the capnography sensor. In yet another example, the indication to exit the intubation mode is based on detecting, from the video data, that a tip of an endotracheal tube has passed through vocal cords. In still yet another example, the indication to enter the intubation mode is based on receiving a selection of a button of the anesthesia machine.

In another aspect, the technology relates to a computer-implemented method for incorporating intubation data into an electronic medical record of a patient. The method includes accessing the electronic medical record of the patient; receiving, from an anesthesia machine, intubation data generated during an intubation procedure of a patient, wherein the intubation data includes patient parameters generated during the intubation procedure and video data, from a video laryngoscope, captured during the intubation procedure; concurrently displaying at least a portion of the intubation data with fillable fields of the electronic medical record; receiving at least one interaction with the electronic medical record during the concurrent display; and storing the electronic medical record based on the at least one interaction.

In an example, concurrently displaying at least the portion of the intubation data includes displaying a video player with the video data. In yet another example, the method further includes executing a query over the intubation data to identify intubation data relevant to at least one of the fillable fields of the electronic medical record. In a further example, the method includes further comprising displaying one or more suggested entries to one or more of the fillable fields based on the identified relevant intubation data. In a still further example, the method further includes automatically filling one or more of the fillable fields with the identified relevant intubation data. In yet another example, the intubation data includes data extracted from the video data including at least one of an endotracheal tube size and a difficulty score for the intubation procedure. In still yet another example, storing the electronic medical record includes storing the video data with the electronic medical record.

In another aspect, the technology relates to an anesthesia machine that includes a display; at least one processor; and memory storing instructions that, when executed by the at least one processor, cause the anesthesia machine to perform operations. The operations include receive sensor data from one or more connected sensors; receive first video data from a video laryngoscope; receive second video data from a non-contact monitoring device (NCMD); based on a first indicator generated from at least one of the sensor data, the first video data, or the second video data, automatically entering an intubation mode, wherein entering the intubation mode includes displaying at least one of the first video data and the second video data on the display; and based on a second indicator generated from at least one of the sensor data, the first video data, or the second video data, automatically exiting the intubation mode.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example anesthesia machine.

FIG. 2 depicts an example video laryngoscope.

FIG. 3A depicts an example view of a patient environment that includes a video laryngoscope in communication with an anesthesia machine.

FIG. 3B depicts an example view of another patient environment that includes a non-contact monitoring device in communication with an anesthesia machine.

FIG. 4A depicts example of content displayed on a display of the anesthesia machine during an intubation procedure.

FIG. 4B depicts another example of content displayed on a display of the anesthesia machine during an intubation procedure.

FIG. 5A depicts a schematic diagram of an example anesthesia machine.

FIG. 5B depicts a schematic diagram of an example non-contact monitoring device.

FIG. 6 depicts a schematic diagram of an example video laryngoscope.

FIG. 7A depicts a method for entering and exiting an intubation mode on an anesthesia machine.

FIG. 7B depicts example method for assessing an intubation based on depth data.

FIG. 8 depicts a method for populating an electronic medical record with data associated with intubation mode.

### DETAILED DESCRIPTION

Patients who require breathing assistance may be connected to a mechanical ventilator via a breathing tube (e.g., an endotracheal tube). In a medical procedure referred to as an intubation, a clinician inserts a breathing tube into the mouth of the patient, past the larynx, and into the trachea. The breathing tube may then be connected to a ventilation system that includes an anesthesia machine, mechanical ventilator, and/or other device for supplying breathing gases (e.g., anesthetic gases, oxygen, etc.) to the patient.

A laryngoscope may be used during intubation to help the clinician manipulate portions of the patient's anatomy, such as the tongue and epiglottis, and obtain a view of the larynx sufficient for inserting the breathing tube into the trachea. To further help visualize the larynx, some laryngoscopes may be configured with a camera system that includes a video camera and light source. A laryngoscope that includes a camera system may be referred to as a video laryngoscope (VL). The video laryngoscope includes an integrated display on which the clinician may view images acquired by the camera.

A video laryngoscope may further include a feature that provides for wireless connection between the video laryngoscope and other medical devices or systems. For example, the video laryngoscope may establish connection with another medical device via an optical-based method (such as to pair with the device), and the video laryngoscope may then communicate with the other medical device using a more robust wireless communication method (e.g., WiFi or Bluetooth). One example pairing and transmission method is described in detail in U.S. Provisional Patent Application No. 63/505,275, titled Video Laryngoscope and Medical Device Wireless Video Transfer, which is incorporated herein by reference in its entirety.

With the technology described herein, the video laryngoscope may pair with an anesthesia machine and may provide video images, still images, and/or other data acquired by the video laryngoscope during intubation to the anesthesia machine. The images and/or data may be viewed on a display associated with the anesthesia machine, such as on an integrated or connected display of the anesthesia machine. In some examples, the display of the anesthesia machine may be used to replicate the view on the display of the video laryngoscope, so that the clinician performing the intubation, and/or other supporting clinicians, may view the video laryngoscope image and other data more easily (on the larger display of the anesthesia machine).

In addition to viewing the image data provided by the video laryngoscope, a clinician performing the intubation may also prefer to view additional health data that may indicate the overall condition of the patient. For example, a patient undergoing intubation may be connected to multiple monitors or sensors, such as a pulse oximeter, which measures the oxygen saturation (e.g., SpO2) of the patient's blood. Performing the intubation may interrupt or disturb the patient's normal breathing and cause a drop in SpO2 over the course of the procedure. The clinician performing the procedure may wish to monitor SpO2, so as to determine whether to continue the intubation or whether to pause the intubation procedure and allow the patient's SpO2 to recover. The clinician may then make another attempt to complete the intubation.

In other examples, the clinician may prefer to monitor other health and/or respiratory parameters that indicate a successfully completed intubation. For instance, the anesthesia system, or elements associated with the anesthesia system, may include a sensor for measuring the carbon dioxide (CO2) concentration of exhaled breathing gases (e.g., a capnometry sensor). When properly positioned, the breathing tube (e.g., endotracheal tube) may allow the exhaled breathing gases to flow past the capnometry sensor, which provides a signal indicating the CO2 concentration. In one example, the capnometry sensor may provide a measure of end-tidal CO2 (e.g., EtCO2). Additional sensors, including one or more non-contact monitoring devices (NCMDs) may also be in communication with the anesthesia machine and/or the video laryngoscope to provide health and/or respiratory parameters during the course of intubation. The clinician may use the CO2 concentration and/or EtCO2 measurement to determine whether the breathing tube is properly positioned in the patient's airway (e.g., in the trachea). In still other examples, the clinician may prefer to observe multiple or alternative health and/or respiratory parameters during the course of the intubation.

The technology disclosed herein facilitates intubation by providing an intubation mode, of the anesthesia machine, in which health and/or respiratory parameters may be viewed on a display of the anesthesia machine concurrently with images acquired by the video laryngoscope and/or the NCMD during intubation. The clinician may cause the anesthesia machine to enter intubation mode via a button press (e.g., a virtual button) or by providing other types of input. The intubation mode may also or alternatively be entered automatically, such as based on the detection of particular triggers from connected sensors, the video laryngoscope, and/or the NCMD. Upon entering intubation mode, the anesthesia machine may start a timer that tracks the amount of time spent in intubation mode. The anesthesia machine may exit intubation mode either by receiving input from the clinician, or the anesthesia machine may automatically exit the intubation mode based on the detection of one or more trigger conditions. In some examples, the anesthesia machine may exit intubation mode based on measured or derived health and/or respiratory parameters, such as those described above (e.g., EtCO2, other respiratory parameters). In other examples, the anesthesia machine may enter and/or exit intubation mode based on algorithmic analysis of video images received from the video laryngoscope and/or the NCMD. This analysis may be provided by an artificial intelligence (AI) or machine learning (ML) feature or processing of the video data captured by the video laryngoscope.

In addition, data may be aggregated by the anesthesia machine during intubation mode and included as part of the patient's electronic medical record (EMR). For example, the clinician may tag acquired images during or after the intubation for inclusion in the EMR, such as images of the patient's airway, a video segment showing a successful (or unsuccessful) intubation, and/or other types of images. The aggregated data may include the value of the intubation timer, the number of attempted intubations, measured parameters recorded before/during/after the intubation, and/or other data associated with the intubation. In some examples, portions of the patient's EMR may be automatically populated with images and/or data acquired during the intubation, which may be reviewed and accepted and/or edited by the clinician. Additional details are now provided via discussion of the included drawings.

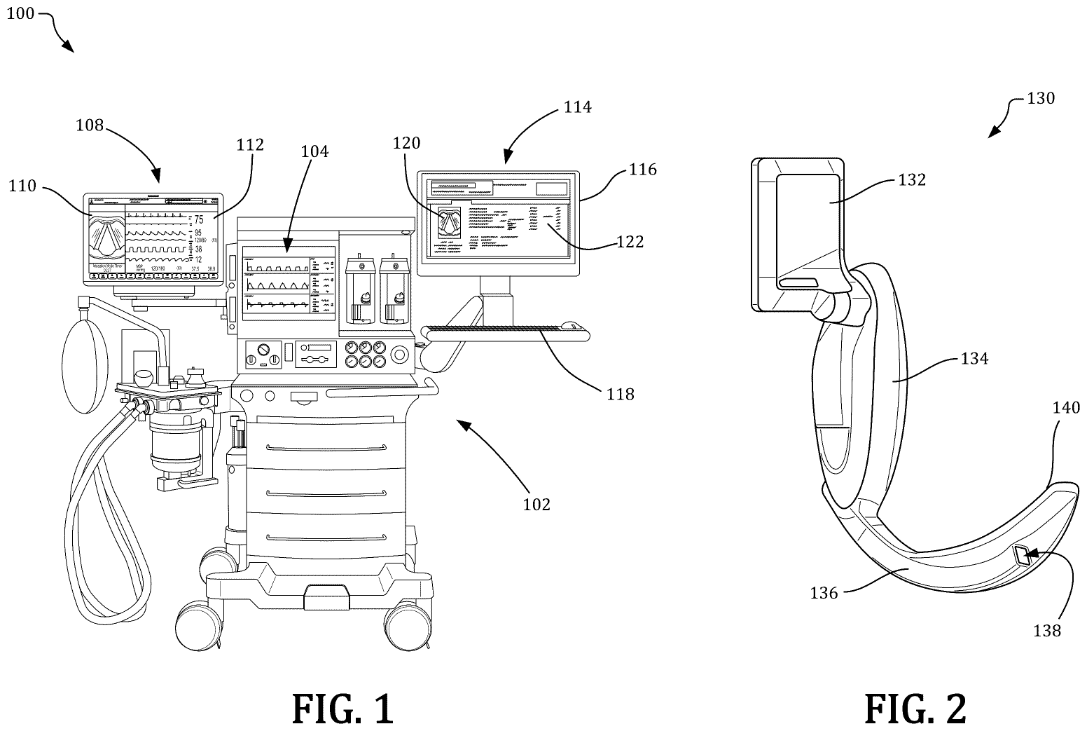

FIG. 1 depicts an example anesthesia machine 100 that includes an intubation mode. The anesthesia machine 100 includes a base portion 102, which may include a cart and other primary controls of the anesthesia machine 100. In addition to the controls of the anesthesia machine 100, the base portion 102 may further include a primary display 104 that displays data related to the anesthesia operations of the anesthesia machine 100. In addition, the anesthesia machine 100 may include a secondary display or multi-parameter-monitor (MPM) display 108.

During normal operation, the MPM display conveys patient parameters based on sensor data collected from various sensors that are connected to the anesthesia machine 100, such as a heart rate sensor, blood pressure sensor, pulse oximeter, capnometer, etc. In addition, a patient terminal 114 may be coupled to, or included in, the anesthesia machine 100. The patient terminal 114 may be in the form of a computer that allows for interaction with the patient's EMR. For instance, the patient terminal 114 may include a terminal display 116 that display the EMR data and a terminal input device 118, such as a keyboard, mouse, touchscreen, etc.

The anesthesia machine 100 provides a supply of breathing gases (e.g., oxygen, nitrous oxide) mixed with a concentration of anesthetic vapor (e.g., isoflurane, sevoflurane). This mixture of gases is then delivered to the patient via a patient circuit that is connected to a patient interface (e.g., mask, endotracheal tube) of the patient. During an intubation procedure, a video laryngoscope is used to position the endotracheal tube (ETT) within the trachea of the patient. During at least part of this procedure, the patient circuit is not connected to the ETT or delivering the medical gases to the patient. For example, prior to intubation, the patient may be provided oxygen and anesthetic via a mask. During the intubation procedure, the mask is removed and the ETT is positioned in the patient with the guidance of the video laryngoscope. During that time, the patient is briefly disconnected from the anesthesia machine. As such, quickly and properly positioning the ETT can be a critical component of preparing the patient for a surgical procedure.

With the technology described herein, the intubation procedure can be assisted and tracked by the anesthesia machine 100. For instance, at the start of the intubation procedure, the anesthesia machine 100 enters an intubation mode. During the intubation mode, the anesthesia machine 100 adjusts the types of data displayed on one or more of the displays of the anesthesia machine 100, and the anesthesia machine 100 tracks patient parameters and the total duration of the intubation procedure. This information may then be used to fill portions of the patient's EMR by transferring such information to the patient terminal 114.

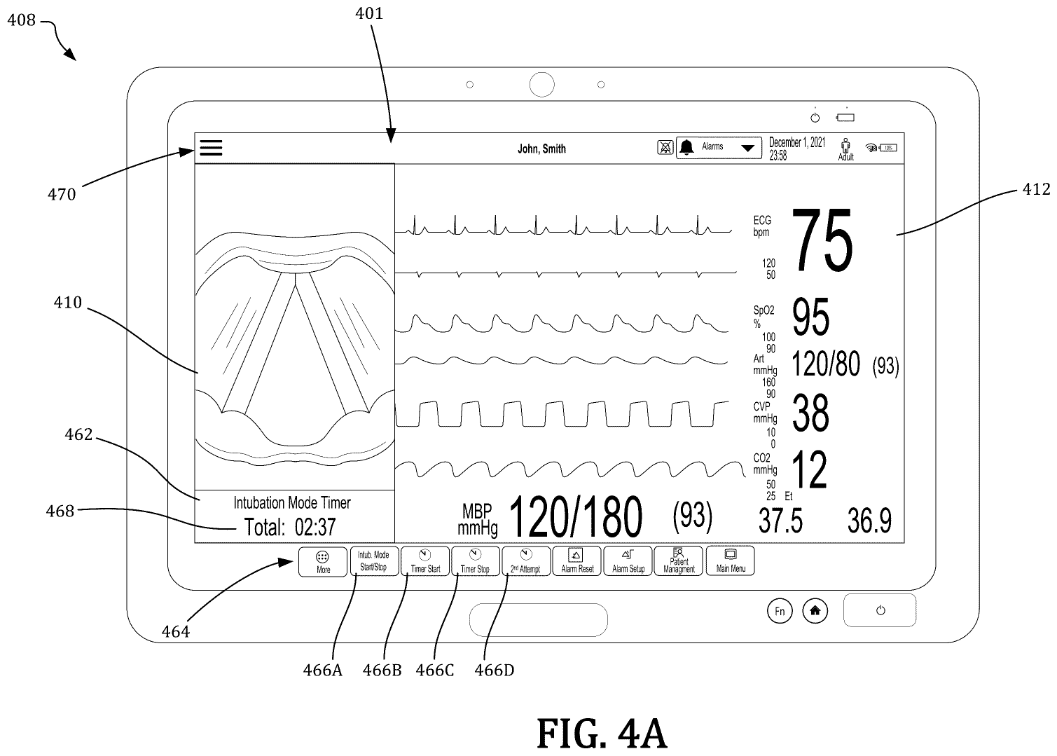

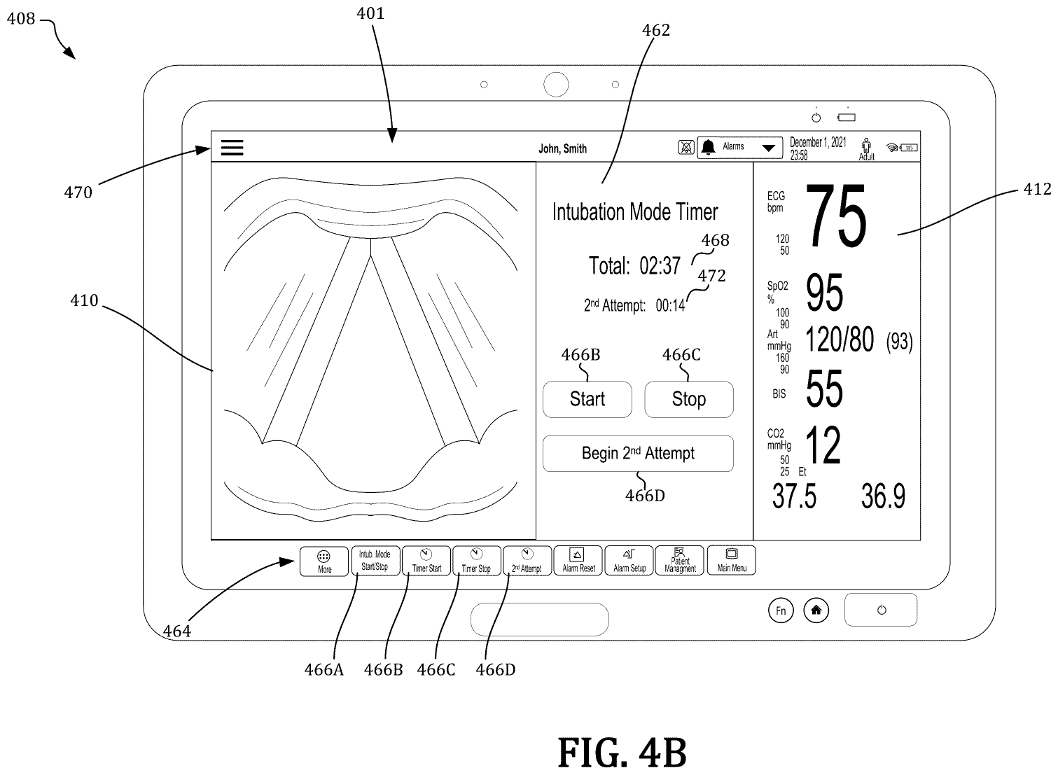

As an example, during the intubation mode, the data displayed on the MPM display 108 may be adjusted from a normal operation mode (e.g., non-intubation mode). For instance, during the normal operation mode of the anesthesia machine 100, the MPM display 108 displays patient parameters from sensors connected to (e.g., in communication with) the anesthesia machine 100. When the anesthesia machine 100 enters the intubation mode, the MPM display 108 displays a video feed from the video laryngoscope (and/or the NCMD) and a subset of the patient parameters that were previously displayed. In an example, the MPM display 108 includes a video laryngoscope segment 110 displaying the video feed from the video laryngoscope and a patient-parameter segment 112 displaying the subset of the patient parameters. The MPM display 108 may also display an intubation timer indicating the current duration of the intubation procedure. Additional discussion of the particular interfaces displayed on the MPM display 108 are discussed further below with reference to FIGS. 4A-4B. While the MPM display 108 is primarily discussed as being adjusted when entering the intubation mode, in other examples another display of the anesthesia machine 100 (e.g., primary display 104, terminal display 116) may be adjusted alternatively or in addition to the MPM display 108 to display the video feed(s) and/or subset of patient parameters.

The data collected during the intubation mode may then be used to populate the patient's EMR via the terminal display 116. For example, the anesthesia machine 100 may cause the automatic population of data in the EMR entries for the intubation procedure. For instance, entries for the EMR may be completed through an Anesthesia Information Management System (AIMS) or similar system. AIMS may be considered a specialty form of EMR that is specifically designed for the unique needs of the anesthesia workflow. For instance, an example AIMS may be able to automatically capture data from the anesthesia machine and patient monitors, such as patient vitals, and populate that data into the EMR for the patient. Current EMR systems and AIMS technology, however, do not utilize video laryngoscope data for the population of the EMR.

With the technology disclosed herein, not only can the anesthesia information and patient parameters collected by the anesthesia machine 100 be automatically incorporated into the EMR, the video laryngoscope data and/or NCMD data received by the anesthesia machine 100 during the intubation mode may also be automatically incorporated into the EMR. For example, a copy of video file received during the intubation mode from the video laryngoscope and/or the NCMD may be stored with the patient's EMR to allow for later access and review. Alternatively or additionally, the video data may be analyzed to extract data that is automatically incorporated into the EMR. For instance, object recognition algorithms may be executed against the video data to identify and/or characterize objects within the video. Such objects may include medical instruments (e.g., the ETT) and/or anatomical objects (e.g., vocal cords). Such extracted information may be useful in populating the EMR portions that are directly related to the intubation procedure, such as fields relating to duration of the intubation procedure, size and type of ETT, laryngoscope blade type and size, use of stylets, use of a video laryngoscope, correct positioning confirmation data, number of attempts, etc. In some examples, this data may be populated into the fields automatically for review by the clinician and/or provided as suggestions to the clinician for data that may be populated within the field. In either case, the total manual entries required and total time to accurately complete the EMR is reduced significantly.

In some examples, the video laryngoscope video data and/or the NCMD video data may be presented in an interactive manner concurrently with the fillable fields of the EMR. For instance, the terminal display 116 may display a video segment 120 and a field segment 122. The video segment 120 may include an interactive video captured by the video laryngoscope and/or NCMD and received by the anesthesia machine 100 during the intubation procedure. The clinician may be able to interact with the displayed video, such as to navigate to a particular point in time during the intubation procedure. In some examples, the video data may be analyzed to identify significant events during the intubation procedure, and the timestamps of those significant events may be tagged in the video file and presented via the video laryngoscope segment of the terminal display 116. For instance, markers or tags may be displayed on the navigation bar of the video playback interface that indicate when significant events occurred, such as a first detection of the ETT, a detection of the vocal cords, a detection that the tip of the ETT has passed through the vocal cords. Accordingly, the clinician may skip to particular time points of interest during the intubation procedure.

FIG. 2 depicts an example video laryngoscope 130. The video laryngoscope 130 includes a handle 134, a display 132, an extension arm 136, and a camera 138. A video laryngoscope blade 140 is also shown as attached to the video laryngoscope 130. When the blade 140 may be attached by sliding the blade 140 over the extension arm 136. The video laryngoscope 130 may be powered by a battery located in the handle 134.

When in use, the blade 140 and the arm 136 are positioned within the patient's mouth and upper airways. The camera 138 positioned at the end of the arm 136 captures images during the intubation procedure as the ETT is inserted past the camera 138 and the blade 140. The captured images may be captured as video. The live video feed may be presented on the display 132 of the video laryngoscope 130. The backside of the display 132 is depicted in FIG. 2, but the display of the video feed is presented on the front side of the display.

In some examples, the clinician may also interact with the video laryngoscope 130 to capture additional data about the intubation procedure. For instance, the video laryngoscope 130 includes one or more input elements, such as a button and/or the display 132 may be a touchscreen. The interactions may cause a certain point of the procedure to be marked or flagged as important. The interactions may also or alternatively cause a still image of the video feed to be captured and stored.

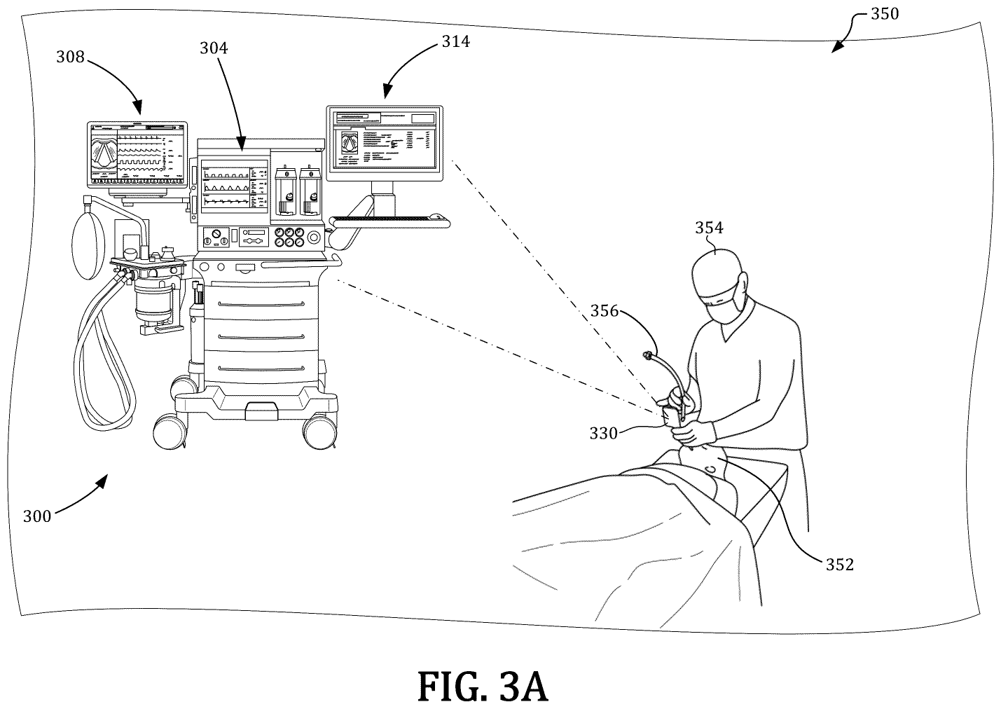

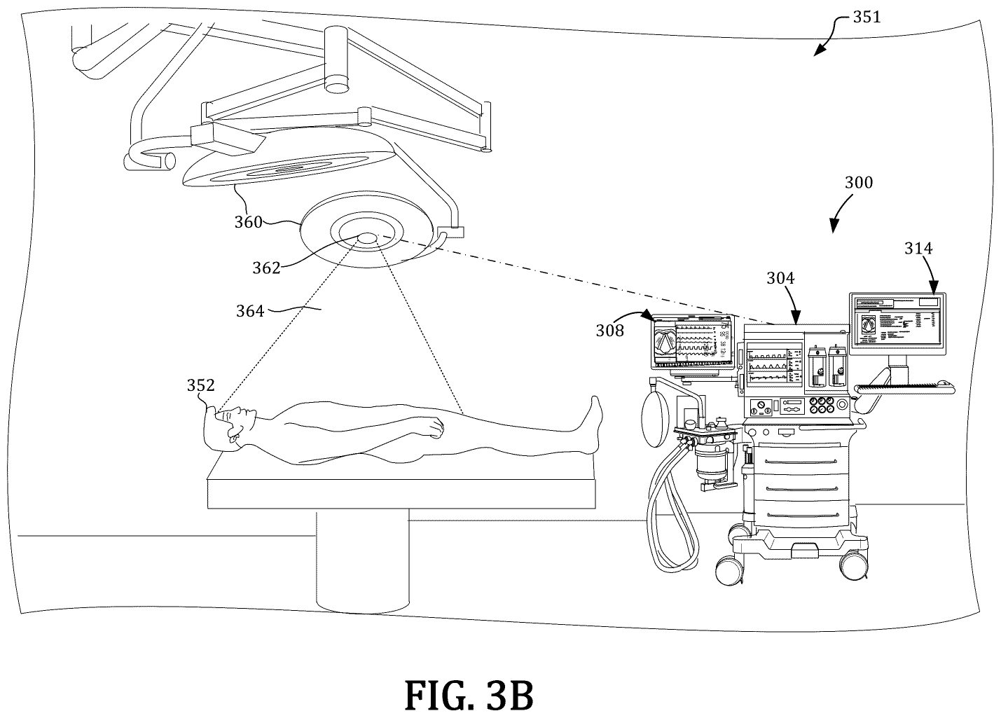

FIG. 3A depicts an example view of a patient environment 350 that includes an example video laryngoscope 330 in communication with an example anesthesia machine 300 during an intubation procedure. The patient environment 350 can be any room or theater where an intubation is being performed, such as a medical suite in a hospital or other care setting, an operating or other procedure room, patient recovery room, an emergency intubation setting (e.g., an ambulance), or other environments. The anesthesia machine 300 may be similar to, or the same as, anesthesia machine 100, depicted in FIG. 1. The video laryngoscope 330 may be similar to, or the same as, video laryngoscope 130, depicted in FIG. 2.

During the intubation procedure, the laryngoscope operator 354 (e.g., clinician) holds a handle of the video laryngoscope 330. Acquired image data is displayed on the display of the video laryngoscope 330. As part of an intubation procedure, the ETT 356 is advanced into the airway of a patient 352 to secure the airway for anesthesia. Accordingly, the operator 354 of the video laryngoscope 330 performs the intubation and directly manipulates the ETT within the patient's airway, and other clinicians in the patient environment assist the laryngoscope operator 354, monitor the condition of the patient 352, prepare or adjust medical equipment in the patient environment 350, and/or wait until the airway is secured to perform other procedures or interventions. As provided herein, the image data can be stored in a memory on the video laryngoscope 330. The image data may be in the form of video data (e.g., a video feed, video stream) and/or may be in the form of still images.

In the example depicted in FIG. 3A, the patient environment 350 includes an anesthesia machine 300, with which the video laryngoscope 330 may be in communication as described further herein. In addition, the anesthesia machine 300 may include, or be connected to, sensors for measuring physiological parameters of the patient during the intubation procedure, such as pulse oximetry sensors, heart rate sensors, ECG, sensors, capnometry sensors, blood pressure sensors, etc. Such data may be displayed on the MPM display 308 of the anesthesia machine 100. Some data may also be displayed on the primary display 304 of the anesthesia machine 300.

The video laryngoscope 330 wirelessly connects the anesthesia machine 300. Once connected, the video laryngoscope 330 transmits video image data to the anesthesia machine 300, where the anesthesia machine 300 displays and/or stores the received video data.

As described in further detail below, connection or pairing of the video laryngoscope 330 with the anesthesia machine 300 may be performed through the use of optical and non-optical signals with little to no input required from the laryngoscope operator 354 or other clinical staff. In an embodiment, when the video laryngoscope 330 is powered on (e.g., in response to a manual selection of a power button), the video laryngoscope 330 goes through an initial pairing process that includes the emission of an optical signal that is received by the anesthesia machine 300. For instance, an optical transceiver of the video laryngoscope 330 emits an optical signal through an optically transparent window of the video laryngoscope 330 such that the optical signal is emitted throughout the example patient environment 350. The anesthesia machine 300 then detects the optical signal from the video laryngoscope 330. For example, the anesthesia machine 300 may also include an optical transceiver within the housing of the anesthesia machine 300. The optical transceiver of the anesthesia machine 300 processes the received optical signal from the video laryngoscope 330, and the anesthesia machine 300 then emits an optical response signal of its own via its optical transceiver. The video laryngoscope 330 receives this optical response signal, and a non-optical connection between the video laryngoscope 330 and anesthesia machine 300 may then be established.

Once the non-optical connection is established between the video laryngoscope 330 and anesthesia machine 300, the video image data (and/or still image data) captured by the video laryngoscope 330, is transmitted by the video laryngoscope 330 to the anesthesia machine 300 via the non-optical connection. Once received by the anesthesia machine 300, the video image data may be displayed and/or stored by the anesthesia machine 300. For example, the anesthesia machine 300 may display the image data on the MPM display 308 during the intubation mode. Additionally or alternatively, the anesthesia machine 300 may store the image data in memory of the anesthesia machine 300 such that the image data may be accessed at a later time after the intubation procedure has been completed.

In some examples, the video laryngoscope 330 may connect to the anesthesia machine 300) using optical and/or electronic elements integrated within the anesthesia machine 300. In other examples, the video laryngoscope may connect to the anesthesia machine 300 by way of an externally connected adapter (not depicted), such as a pluggable adapter, module, dongle. For example, the optical and non-optical elements used for connecting the anesthesia machine 300 to the video laryngoscope 330 may be integrated in a portable adapter that may be detachably connected to the anesthesia machine 300.

In further examples, the patient terminal 314 may include an exterior port for receiving the portable adapter. In such examples, the adapter may be plugged directly into the patient terminal 314 and video data from the video laryngoscope 330 may be received by the patient terminal without the video data having to be transferred through or via other components of the anesthesia machine 300. In such examples, during the intubation mode, the video data from the video laryngoscope 330 may be displayed on the patient terminal 314 alternatively (or in addition to) the MPM display 308.

In still other examples, the MPM display 308 may include an exterior port for receiving the portable adapter. In such examples, the video data from the video laryngoscope 330 may be received by the MPM display 308 without have to be transferred through or via other components of the anesthesia machine 300. During the intubation mode, the MPM display 308 accesses the video data received from the video laryngoscope 330, via the adapter, and displays the video data.

FIG. 3B depicts an example view of another patient environment 351 that includes a NCMD 362 in communication with an anesthesia machine 300. The anesthesia machine 300 may be substantially the same as the anesthesia machine 300 in FIG. 3A. The patient 352 may also be the same and, in some examples, the patient 352 may be undergoing an intubation procedure or currently be intubated. The tubing and/or other physical connections are not depicted for clarity. The patient environment 351 may be similar to or the same as the patient environment 350. For instance, the patient environment 351 can be any room or theater where an intubation is being performed, such as a medical suite in a hospital or other care setting, an operating or other procedure room, patient recovery room, an emergency intubation setting (e.g., an ambulance), or other environments.

In the example depicted, the NCMD 362 has been integrated into a surgical light 360 that is mounted above the patient 352. In other examples, however, the NCMD 362 may be provided in different positions within the patient environment 351, such as on a movable cart, the operating table, or other fixtures within the patient environment 351. By mounting the NCMD 362 within a surgical light 360, however, some additional benefits may be achieved. For example, the NCMD 362 provides the direct benefit of fewer wires or cables connected to the patient 352. By mounting the NCMD 362 to the surgical light 360, power can be provided to the NCMD 362 so that power cables near the patient or along the floor of the patient environment 351 can also be avoided. Cable-based connections (e.g., Ethernet) may also be provided through ceiling ports near the surgical light 360 to further avoid additional cables along the floor of the patient environment 351.

The NCMD 362 captures images of the patient 352, such as during an intubation procedure, while the patient 352 is intubated, and/or while the patient 352 is being extubated. In some examples, the NCMD 362 also emits an optical pattern 364, which causes a projection of light onto the patient 352. For instance, the optical pattern 364 may be an infrared grid or lattice pattern than is detected by one or more cameras of the NCMD 362. In some examples, the NCMD 362 includes at least two cameras to allow for stereoscopic imaging of the patient 352. Accordingly, the NCMD 362 in some examples includes, or is, a depth-sensing camera system.

The NCMD 362 provides depth sensing capabilities that allows for sensing changes in the patient 352 without contacting the patient 352. For instance, the NCMD 362 may capture a sequence of images over time. Within each image, the depth sensing capabilities detect a distance between the camera and objects within its field of view. For instance, a depth value may be determined for each image. A grouping of pixels may define a region of interest (ROI), which may be correspond to a particular portion of the patient 352 and/or a portion of a surgical tool, such as the example video laryngoscope 330 or the ETT 356. The depth information may be used in combination with other signals from the pixel (such as red, green, blue (RGB) signals) to identify and/or classify objects within the captured images. The depth information and/or other signals may also be used to identify changes in the patient 352 and/or the surgical tools. For instance, breathing characteristics of the patient may be identified based on depth changes of the chest region of the patient 352, as described in U.S. Patent Publication No. 2023/0410343 and the other applications discussed therein. As an example, the breathing characteristics may include tidal volume, minute volume, and/or respiratory rate, among others. The movements or changes depths within the chest region also be used to detect various breathing abnormalities, such as apnea, rapid breathing (tachypnea), slow breathing, intermittent or irregular breathing, shallow breathing, obstructed and/or impaired breathing, and others.

As an example, the movement of the chest may be analyzed based on the depth changes of the chest. The repetitive nature of the chest movement over time may be used to determine the respiratory rate of the patient. The lack of chest movement (e.g., a respiratory rate of zero) may also be indicative of the beginning of an apneic period associated with the intubation procedures discussed herein.

After the intubation has been completed and breathing gases are flowing into the lungs of the patient, chest movement may be again detected by the NCMD 362. The movement of the chest may be indicative of the completion of the intubation procedure.

Further, the way or manner that the chest and/or abdomen moves may also be indicative of whether the intubation procedure has been properly performed. For example, during the intubation procedure, one potential error is to position the ETT in the esophagus rather than in the trachea. In such an example, breathing gases flow into the stomach rather than into the lungs of the patient. As a result, an inflation of the abdomen (corresponding to the position of the stomach) is detected by the NCMD 362. To do so, the NCMD 362 identifies the torso region of the patient and partitions the torso region into a chest region and an abdomen region. When a change in depth of the abdomen region is identified (e.g., indicating an inflation of the abdomen) without an inflation of the chest (or a smaller inflation of the chest), such an identification may be indicative of an improper intubation into the esophagus.

Detection of inflation of the stomach (e.g., increasing volume in the abdomen) may also be indicative of other potential issues where gas has been passed into the stomach, even without an esophageal intubation. For example, when the ETT or other airway device is not properly positioned or sealed, gas may travel to other portions of the body (such as the stomach) rather than out of the nose or mouth of the patient. Anytime gas is passed in such a manner into the stomach, a risk of aspiration is increased. As such, detection of inflation of the stomach and/or abdomen is useful in preventing such aspiration events.

Different subregions of the chest may also be analyzed to determine whether an intubation has been properly performed. Another example of an improper intubation is when the distal end of the ETT is placed beyond the trachea and into one of the right main-stem bronchus or the left main-stem bronchus. In such cases, the lungs inflate unevenly or asymectrically. For example, when the ETT is placed in the right main-stem bronchus, the right lung inflates more than the left lung (if the left lung inflates at all). The NCMD 362 detects such asymmetrical inflation by analyzing the depth changes of the left half of the chest as compared to the depth changes of the right half of the chest. Such an analysis may be performed by partitioning the chest region into a left half and a right half. The depth changes of the right half may then be compared to the depth changes of the left half. If the depth changes between the right half and the left half differ by more than a threshold amount, an improper main-stem intubation may be detected.

In some cases, however, a main-stem intubation is actually desired. For instance, during intubations of neonatal patients or for particular medical purposes (e.g., treatment of one lung), an intubation into a single lung may be desired. In such cases, the intent is to locate the distal tip of the ETT beyond the carina and into the right main-stem bronchus or the left main-stem bronchus. The NCMD 362 also detects whether such a main-stem intubation has been performed correctly. For instance, similar to the procedure discussed above, the chest region may be portioned into a left half and a right half. The changes in depth between the left half and the right half may then be compared. Based on the different between the changes in depth, a determination is made as to whether the correct main-stem intubation was completed correctly. For instance, if the left half rises at least a threshold amount more than the right half, a left main-stem intubation may be confirmed.

Further determinations may also be made based on the depths detected by the NCMD 362. For instance, in some intubations, such as intentional main-stem intubations, the distal tip of the ETT may also travel further than intended, such as into a further generation of bifurcations in a lung. When the distal dip of the ETT is beyond an intended bifurcation (e.g., further into the lung), only a portion of the lung is inflated, such as a lower half of the lung. This may be undesirable where the entire lung was intended to be ventilated. The NCMD 362 protects against this condition by detecting the asymmetrical inflation of a single lung. To do so, the NCMD 362 may first partition the chest region into two halves (representing the left lung and the right lung). The two halves are then partitioned again into two regions each—one representing an upper part of the respective lung and one representing a lower part of the respective lung. As such, four subregions are formed within the chest region: a first subregion corresponding to the upper portion of the left lung, a second subregion corresponding to the lower portion of the left lung, a third subregion corresponding to the upper portion of the right lung, and a fourth subregion corresponding to the lower portion of the right lung. The change in depth of the subregion for an upper portion of the lung may then be compared to the change in depth of the subregion for a lower portion of the lung. If the changes in depth differ by more than a threshold, an improper intubation may be detected (where the intended intubation was a main-stem intubation).

In some examples, objects may also be detected within the images captured by the NCMD 362. For example, the video laryngoscope and/or the ETT may be detected within the video feed captured by the NCMD 362. Similarly, object detection may be used to detect the face and/or mouth of the patient 352. A determination may be made as to when the video laryngoscope and/or ETT enters the mouth or face of the patient 352. Such a determination may be used as an indicator that the intubation procedure has been initiated or is at a particular intermediate state during the procedure.

Another object that may be detected within the images captured by the NCMD 362 is the mask that is used to temporarily ventilate the patient prior to intubation. As discussed above prior to intubation, the patient 352 may be provided oxygen and anesthetic via a mask. During the intubation procedure, the mask is removed and the ETT is positioned in the patient 352 with the guidance of the video laryngoscope. The removal of that mask may be detected from the video captured by the NCMD 362. In some examples, the object detection and/or classification may be performed on depth data from the NCMD 362. In other examples, the object detection and/or classification may be performed on non-depth video signals, such as a red-green-blue (RGB) video signals (and/or the individual frames thereof). Detection of removal of the mask is indicative of the start of the apneic period because the patient is no longer receiving breathing gases from another source, such as a manual bag and/or the anesthesia machine 300.

The data collected and/or generated by the NCMD 362 may be communicated to the example anesthesia machine 300 for display, incorporation into an EMR, and/or for activating and/or deactivating intubation modes. In some examples, the NCMD 362 may establish a connection with the example anesthesia machine 300 in a similar manner as the video laryngoscope 330 pairs with the anesthesia machine 300. For instance, optical and/non-optical signals may be used to establish the connection. As an example, an initial pairing process may include an optical pairing signal being generated by the NCMD 362 that is received by the example anesthesia machine 300. Based on the optical pairing signal, a non-optical connection may then be established between the NCMD 362 and the anesthesia machine 300 to allow the NCMD 362 to transmit data to the anesthesia machine 300 via the non-optical connection. In other examples, the connection between the NCMD 362 and the anesthesia machine 300 may be established in different manners. For instance, when the NCMD 362 is a more permanent fixture in the operating room, such as when integrated into the surgical light 360, the NCMD 362 may have a wired or wireless connection to the hospital network (e.g., a local area network, Wi-Fi network). The example anesthesia machine 300 may be similarly connected to the hospital network. Based on addresses (e.g., Internet Protocol (IP) address, media access control (MAC) address) of the NCMD 362 and the anesthesia machine 300, a connection between the NCMD 362 and the anesthesia machine 300 may be established such that the NCMD 362 is able to send data to the example anesthesia machine 300.

FIG. 4A depicts example of content displayed on a display 408 of the anesthesia machine during an intubation procedure. The display 408 may be an MPM display of the anesthesia machine in some examples. In other examples, the display 408 may be a different display, such as the terminal display and/or the primary display.

The content shown on the display 408 may be initiated upon the anesthesia machine 100 entering an intubation mode. As such, the displayed content may be referred to herein as an intubation-mode interface (IMI) 401. The intubation-mode interface 401 includes a plurality of different segments, such as a video laryngoscope segment 410 and a patient-parameter segment 412. Additional segments may include a timer segment 462 with an intubation timer 468, a header segment 470, and a virtual button segment 464.

The video laryngoscope segment 410 displays the video feed that is received from the video laryngoscope. In some examples, the video laryngoscope segment 410 (and the video feed) occupies at least 25% of the display area of the respective display. By providing the video feed from the video laryngoscope on the display, other clinicians within the operating room are able to view the progress of the intubation and be ready with next steps or assistance to be performed based on the progress of the intubation. When the intubation mode is exited or ended, the video laryngoscope segment 410 is removed from the display, and the display area that previously was filled by the video laryngoscope segment 410 may be populated with additional or expanded patient parameter data. In some examples, the video data captured by the NCMD may be displayed additionally and/or alternatively from the video data from the video laryngoscope. The NCMD video data may be displayed in the video laryngoscope segment 410 and/or another segment. In some examples, the video feed may be manually toggled between the video laryngoscope data and the NCMD video data.

The patient-parameter segment 412 includes patient-parameter data that is pertinent to the intubation procedure. The patient-parameter data that is displayed during the intubation mode may be different than the patient-parameter data when the anesthesia machine is not in the intubation mode. For instance, some patient data may be removed from the display when the intubation mode is entered. In some examples, the patient-parameter data that is displayed during the intubation mode may include data such as electrocardiogram (ECG) data, SpO2 data, heart rate, blood pressure, temperature depth of anesthesia level (e.g., bispectral (BIS) index data), physiological pain response data (e.g., nociception levels or data, such as Nociception Level (NOL)), and/or neuromuscular blockade data (e.g., Train-of-Four). This data may be shown as values and/or as waveforms, as depicted in FIG. 4A. In some examples, the patient-parameter segment 412 may occupy between 25-75% of the available screen area. The patient-parameter segment 412 may also be displayed adjacent the video laryngoscope segment 410.

The intubation-mode interface 401 may also include a timer segment 462 that includes an intubation timer 468. The timer 468 is activated/started when the intubation mode is activated or entered. For instance, when a selection is received to enter the intubation mode (or trigger criteria is identified to automatically enter the intubation mode), the timer 468 is started. The timer 468 provides a real-time view of the duration of the current intubation procedure. The timer 468 stops when the intubation mode ends or is exited, and the duration of the intubation procedure is recorded based on the time of the timer 468 at the end of the intubation mode.

The virtual button segment 464 includes a set of virtual buttons associated with the intubation mode, such as virtual buttons 466A-D identified in FIG. 4A. For example, a start/stop button 466A may be included that can be selected to enter or exit the intubation mode. In some examples, separate controls for the timer 468 may be provided, such as a timer start button 466B and a timer stop button 466C. In such examples, the timer 468 may be controlled separately from the entering and exiting of the intubation mode. Such examples may be useful where a confirmation of the intubation mode operation is desired prior to starting the actual intubation procedure. A second attempt button 466D may also be provided in the virtual button area 466D. This second attempt button 466D may be selected when a second attempt at intubation is being performed. Selection of the second attempt button 466D causes the anesthesia machine to record the occurrence of a second attempt. In addition, a second timer or sub timer (similar to a lap timer) may be started to record the duration of the second intubation attempt. Other virtual buttons may also be present that may be selected to control other operations of the intubation mode and/or configuration settings of the anesthesia machine.

The header segment 470 may be used to display patient information, alerts, time of day, general purpose status, etc. For instance, a patient name and patient type (pediatric, adult) may be displayed. The status of sensors may also be displayed. The alarm state may also be displayed. In some examples, activation of the intubation mode silences all alarms. The status of those alarms, however, may still be displayed, such as in the header segment.

FIG. 4B depicts another example of content displayed on a display of the anesthesia machine during an intubation procedure. For instance, FIG. 4B depicts another example of an intubation-mode interface 401. In the example intubation-mode interface 401 depicted in FIG. 4B, the waveform portion the patient-parameter segment 412 has been replaced with an enlarged version of the timer segment 462. In the example depicted, the timer segment includes a total duration timer 468 as well as a second attempt timer 472. The second attempt timer may be displayed and started when a second attempt button 466D is selected. The second attempt timer 472 indicates the duration of a second attempt at intubation. For example, a clinician may stop a first attempt, re-oxygenate the patient through a bag and mask, and then start a new attempt. At the start of the new attempt, the second attempt button 466D may be selected to start the second attempt timer 472.

In the enlarged example of the timer segment 462, a timer start button 466B, a timer stop button 466C, and second attempt button 466D may be displayed within the timer segment 462 alternatively or in addition to the virtual button segment 464. In some examples, an intubation mode start/stop button 466A may also be included within the timer segment 462.

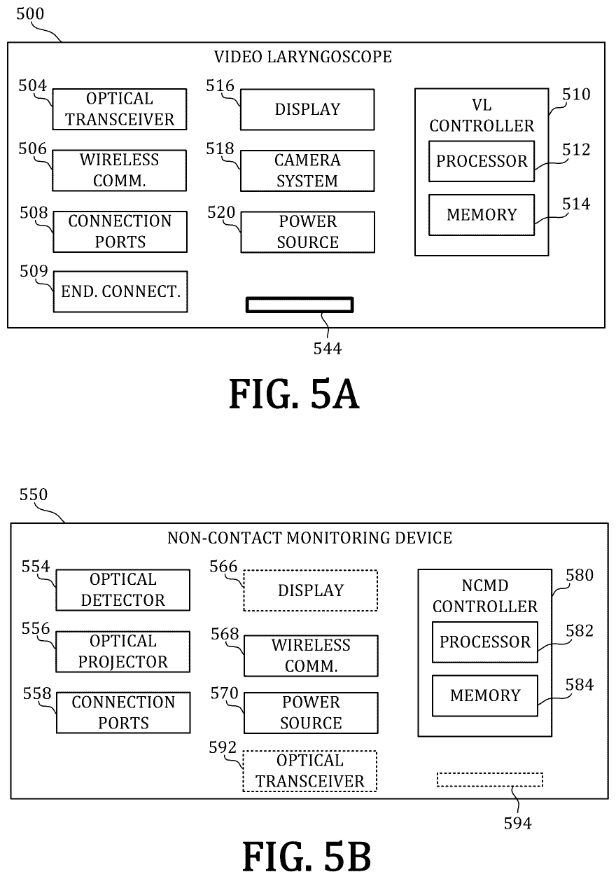

FIG. 5A depicts a schematic diagram of an example video laryngoscope 500, which may be the same as, or similar to, example video laryngoscope 130. The video laryngoscope 500 includes an optical transceiver 504 for transmitting and receiving the optical signals discussed herein. For example, the optical transceiver 504 may be a type of infrared (IR) transceiver or may be capable of transmitting and receiving optical signals in the visible wavelength. The video laryngoscope 500 includes an optical window 544 formed in the housing of the video laryngoscope 500 to allow for the optical signals to reach the optical transceiver 504 and exit the video laryngoscope 500.

The video laryngoscope 500 further includes a wireless communication device 506. The wireless communication device 506 may be a wireless transceiver that is configured to establish wireless communication in a non-optical frequency. By way of example, the wireless communication device 506 may be configured to communicate using the IEEE 802.15.4 standard, and may communicate, for example, using ZigBee, WirelessHART, or MiWi protocols. Additionally or alternatively, the wireless communication device 506 may be configured to communicate using the Bluetooth standard or one or more of the IEEE 802.11 standards or similar communication techniques. In some examples, the video laryngoscope 500 also include one or more connection ports 508. In examples, the connection ports 508 may include one or more external ports for establishing a wired connection.

In some examples, the video laryngoscope 500 may also receive images captured by an external endoscope camera that is connected to the video laryngoscope 500 via an endoscope port 509. Image data acquired by the external endoscope camera may be transmitted to the other devices discussed herein, such as the anesthesia machine, along with image data acquired by the video laryngoscope camera system 518.

The video laryngoscope 500 also includes a display 516, camera system 518, and power source (e.g., battery) 520. The camera system 518 includes a camera for imaging the patient's airway and a light source that illuminates the field-of-view (FOV) of the camera. The light source may be a type of LED, lamp, or other type of light-emitting element. The camera includes an imaging sensor, such as a charge-coupled device (CCD), complementary metal-oxide-semiconductor (CMOS), or other type of sensor.

The display 516 may be any of a variety of display technologies, such as liquid crystal display (LCD), light emitting diode (LED), organic light emitting diode (OLED), or other display technology. In examples, the display 516 may be a touch-sensitive display (e.g., a capacitive touch-sensitive display) capable of receiving input from a user. Aspects of the operation of the video laryngoscope 500 may also be configured via the display 516, such as video image display preferences and other configurable settings of the video laryngoscope 500. In one example, a clinician may tag or mark a video image by providing input via the display 516. For instance, a clinician may provide input through the display 516 to indicate a video segment of interest.

Video images acquired by the video laryngoscope camera system 518 may be displayed on the display 516 or may be combined with images acquired by an attached endoscope and displayed simultaneously. For example, the display 516 may be capable of providing split screen, picture-in-picture, or other method for simultaneously displaying video images.

The video laryngoscope 500 includes a controller 510 that includes one or more processors 512 and memory elements 514. The processor 512 may include one or more general purpose processors, microprocessors, microcontrollers, graphics processing units (GPUs), digital signal processors (DSPs), and/or other programmable circuits. In examples, the processor 512 may include any combination of commercially available components, and/or custom or semi-custom integrated circuits, such as application specific integrated circuits (ASICs). The processor 512 may include elements needed for control or communication with the display 516, camera system 518, wireless communication device 506, optical transceiver 504, and/or other elements of the video laryngoscope 500.

The processor 512 may perform control, interface, communication, or other processing functions by executing instructions that are stored in the memory 514. For instance, the memory 514 may store instructions that, when executed by the processor 512, cause the elements of the video laryngoscope 500 to perform operations described herein. In one example, the memory 514 may store portions of one or more algorithms associated with intubation mode as described below. In another example, the processor 512 and memory 514 may control the pairing process between the video laryngoscope 500 and anesthesia machine. The memory 514 may include random access memory (RAM), read-only memory (ROM), electrically erasable programmable read-only memory (EEPROM), flash memory or other memory technology. The processor and memory may also perform the AI functions (e.g., object detection in images) discussed herein. For instance, the trained AI model(s) may be stored in the memory 514 of the video laryngoscope 500 and executed locally by the processor 512 of the video laryngoscope 500.

FIG. 5B depicts a schematic diagram of an example NCMD 550, which may be the same as, or similar to, example NCMD 362 discussed above with respect to FIG. 3B. The example NCMD 550 includes an optical detector 554, an optical projector 556, one or more connection ports 558, a display 566, a wireless communication device 568, and a power source 570. The example NCMD also includes an NCMD controller 580 that includes a processor 582 and memory 584. The NCMD 550 may also further include an optical transceiver 592 and an optical window 594.

The optical detector 554 may include one or more cameras (e.g., optical sensors, Complementary Metal Oxide Semiconductor (CMOS) sensors, Charge Coupled Device (CCD) sensors) for capturing images of the patient. The optical detector 554 may include at least two cameras to allow for stereoscopic image capture, which in turn allows for the depth detection features discussed herein. The optical detector 554 may also include Time-Of-Flight (ToF) sensors and/or light detection and ranging (LiDAR) sensors for measuring depth changes in the patient. Structured light sensing technology may also be implemented for determining the depth changes of the patient. In examples that rely on light projection for the depth sensing (e.g., structured light, ToF, LIDAR), the NCMD 550 also includes an optical projector 556. The optical projector 556 projects (e.g., transmits) the light signals onto the field of view of the optical detector 554. Such light signals may differ based on the implemented depth-sensing technology. For instance, when structured light sensing is implemented, the optical projector 556 my project a grid or defined pattern of light outside of the visible spectrum, such as projected a grid of infrared light over the patient. The optical detector 554 then detects the projected light as well as capturing an image of the patient in some examples. The images may be consecutively captured as part of a video feed captured by the NCMD 550.

The NCMD 550 may also include connection ports 558, the wireless communication device 568, and the power source 570. These components may be substantially the same as the connection ports 508, the wireless communication device 506, and the power source 520 of the video laryngoscope 500 discussed above. For instance, the NCMD 550 may be powered by a battery in some examples and/or powered by a wired power connection, such as when the NCMD 550 is integrated into a surgical light. The connection ports 508 for the NCMD 550 may also be configured for wired connections to networks, such as through Ethernet connections.

In some examples, the NCMD 550 further includes a display 566. The display 566 may provide images captured by the NCMD 550. The display may also provide depth measurements captured by the NCMD 550. Alternatively or additionally, the determinations and/or insights generated from the data captured by the NCMD 550 may be displayed on the display 566. In other examples, the display may be omitted, and the data is displayed on the anesthesia machine or other suitable display.

In some examples, the NCMD 550 also includes an optical transceiver 592 and an optical window 594. The optical transceiver 592 transmits and receives the optical pairing signals discussed herein. For example, the optical transceiver 594 may be a type of infrared (IR) transceiver or may be capable of transmitting and receiving optical signals in the visible wavelength. The optical window 594 is formed in the housing of the NCMD 550 to allow for the optical pairing signals to reach the optical transceiver 592 and exit the NCMD 550.

The NCMD 550 also includes the NCMD controller 580, which includes a processor 582 and the memory 584. The processor 582 may be similar to, or the same as, processor 512 described above. The processor 582 may perform control, interface, communication, or other processing functions by executing instructions that are stored in the memory 584. The memory 584 may be similar to, or the same as, memory 512 described above. The memory 584 may store instructions, that when executed by the processor 582, cause the NCMD 550 or elements thereof to perform the operations described herein.

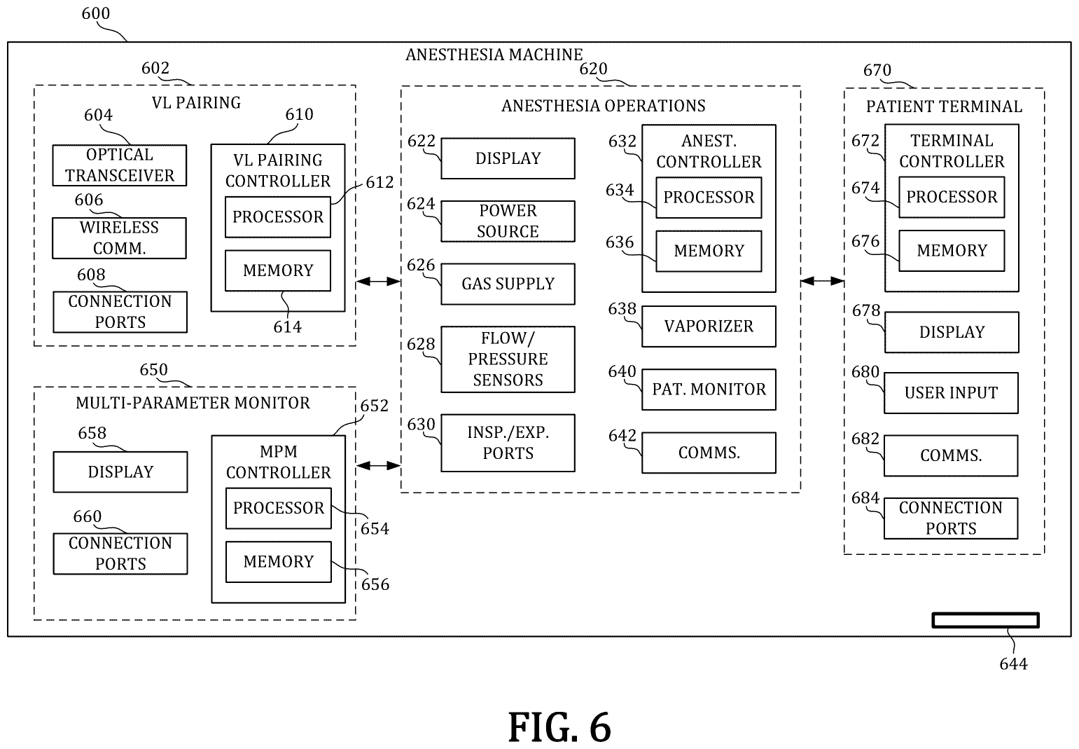

FIG. 6 depicts a schematic diagram of an example anesthesia machine 600. The anesthesia machine 600 includes several different sections, including a video laryngoscope pairing section 602, an anesthesia operations section 620, a MPM section 650, and a patient terminal section 670.

The components of the pairing section 602 may be positioned on, or connected to, a first circuit board or a first set of circuit boards (e.g., printed circuit board assembly (PCBA)), and the components of the anesthesia operations section 620 may be positioned on, or connected to, a second circuit board or second set of circuit boards. In examples, the hardware and/or circuitry of the pairing section 602 is integrated and/or non-removable from the medical device. For instance, the components and/or circuit boards may be physically attached inside the housing of the medical device via screws, adhesives, solder, or other attachment means. The components are non-removable in that they are not intended to be removed or detached by a user (with the limited exception of for repair or replacement during maintenance of the medical device). In the example depicted, the hardware and/or circuitry of the pairing section 602 are not included in any type of removable dongle, plug, or other type or removable or detachable component. The hardware and/or circuitry of the anesthesia operations section 620 are similarly integrated and non-removable from the medical device. In other examples, however, the components of the video laryngoscope pairing section 602 may be included in a removable module, relay, plug, or other similar component that may be removable connected to the anesthesia machine 600.

The pairing section 602 includes an optical transceiver 604, a wireless communications device 606 for non-optical communication, and one or more connection ports 608 to interface with the components of the anesthesia operations section 620. The anesthesia machine 600 may also include an optical window 644 formed in the housing of the anesthesia machine 600 to allow for the optical signals to reach the optical transceiver 604 and exit the anesthesia machine 600. The optical transceiver 604 may be a type of infrared (IR) detector and/or transmitter as described above. The wireless communications device 606 may be the same or similar as wireless communications device 506 described above.

The pairing section 602 also includes a pairing controller 610 that includes one or more processors 612 and hardware memory 614. The processor 612 and memory 614 control the pairing process with the video laryngoscope but may not control the anesthesia operations of the anesthesia machine 600.

While the pairing section 602 is primarily described as being used for pairing the video laryngoscope with the example anesthesia machine 600, in some examples, the pairing section 602 may also be used to pair with the NCMD in examples where the NCMD has optical pairing capabilities similar to the video laryngoscope. In other examples, an additional pairing section may be incorporated into the example anesthesia machine 600. The additional pairing section may be substantially the same as the video laryngoscope pairing section 602 but dedicated to pairing with the NCMD. The anesthesia operations section 620 includes a display 622, a power source 624, a gas supply 626, flow and/or pressure sensors 628, inspiratory and/or expiratory ports and valves 630, a vaporizer 638, and/or patient monitoring components 640. The anesthesia operations section 620 also includes an anesthesia controller 632 that includes one or more processors 634 and hardware memory 636. The anesthesia controller 632 controls the anesthesia operations but may not control or perform operations relating to the pairing of the anesthesia machine 600 with a video laryngoscope. The anesthesia operations section 620 also includes communication device(s) 642 for communicating with devices other than the video laryngoscope. For instance, the communication device(s) 642 may provide for Internet or other networked communications, such as to a medical database or monitoring station.

By having some duplicate components (e.g., processors, memory, communication devices), the primary functions of the anesthesia machine 600 may be segregated from the pairing functions. As such, any operations relating to pairing are less likely to potentially interfere with the life-saving operations of the anesthesia machine 600. However, in other examples, only a single set of processors, memory, and/or communication devices may be present in the anesthesia machine 600 that control both the pairing operations and the anesthesia operations. Such examples reduce the need for duplicate hardware, which ultimately conserves resources and provides for more efficient medical devices.

The MPM section 650 includes a display 658, connection ports 660, and an MPM controller 652 that includes a processor 654 and memory 656. The connection ports allow for the connection of external patient sensors that provide data for displaying the patient parameters discussed herein. Such connection ports 660 may be wired or wireless depending on the sensor types and capabilities.

The patient terminal section 670 includes a terminal controller 672 including a processor 674 and memory 676. The patient terminal section 670 further includes a display 678, a user input device 680 (e.g., keyboard, mouse, touchscreen), communication devices 682, and connection ports 684. The communication device(s) 642 may provide for Internet or other networked communications, such as to a medical database or monitoring station. The connection ports 684 may receive wired connections. For instance, in some examples where the components of the video laryngoscope pairing section 602 are included as a pluggable module, the module may be plugged into one of the connection ports 684 of the patient terminal section 670.

The processors 612, 634, 654, 674, may be similar to, or the same as processor 512 described above. The processors 612, 634, 654, 674 may perform control, interface, communication, or other processing functions by executing instructions that are stored in the memory 614, 636, 656, 676, respectively. The memory 614, 636, 656, 676 may be similar to, or the same as, memory 514 described above. The memory 614, 636, 656, 676 may store instructions, that when executed by the respective processors 612, 634, 654, 674, cause the anesthesia machine 600 or elements thereof to perform the operations described herein.

One or more of the memories, such as memory 636, may store data associated with intubation mode, such as the patient parameters during the intubation mode, the duration of the intubation mode, the video data received by the video laryngoscope during the intubation mode, and/or video data received from the NCMD. This data may then be transferred to the patient terminal section 670 for inclusion in the EMR. The EMR data may be stored in memory 676 of the patient terminal section 670. In some examples, the EMR data is stored temporarily in the memory 676 of the patient terminal section 670 until the data is uploaded to a server for storage.

In some examples, the AI/ML models that are applied to the video feed to detect objects, events, and/or patterns may be stored on one or more of the memories, such as memory 636, of the anesthesia machine 600. In such examples, the analysis of the video feed from the video laryngoscope and/or the NCMD may be performed by the anesthesia machine 600 to extract data for inclusion in the EMR and/or to start or stop the intubation mode.

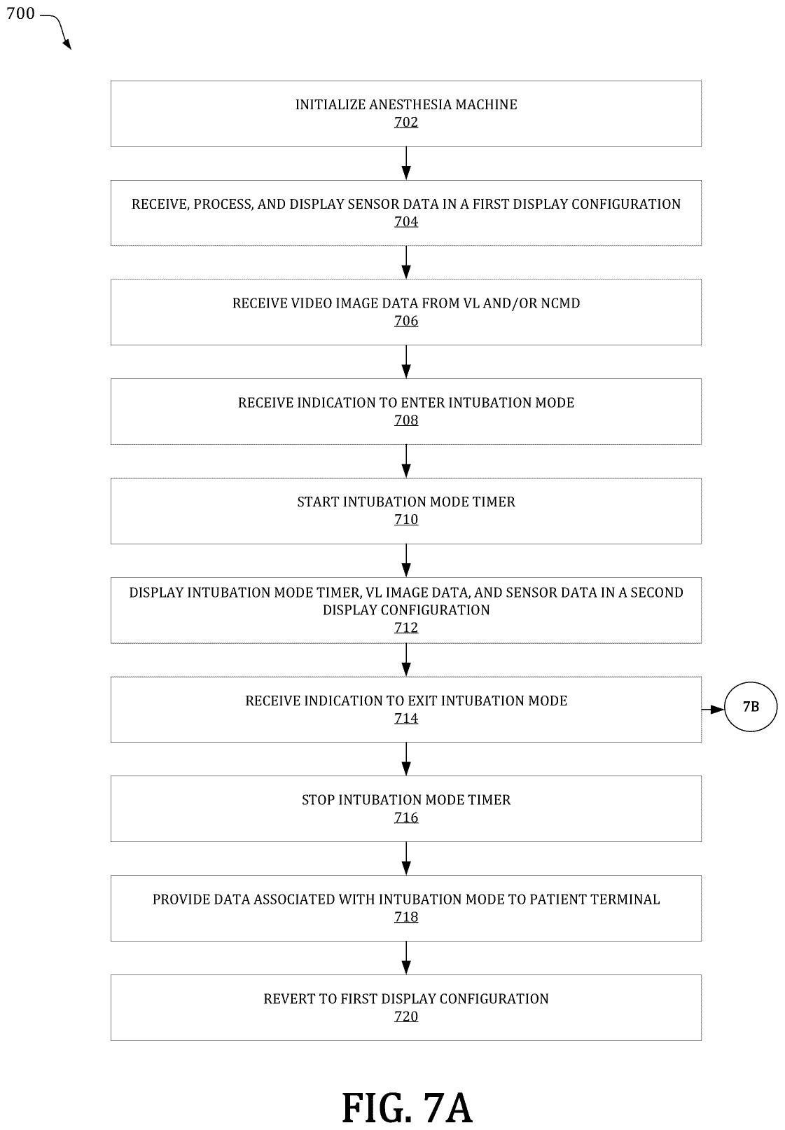

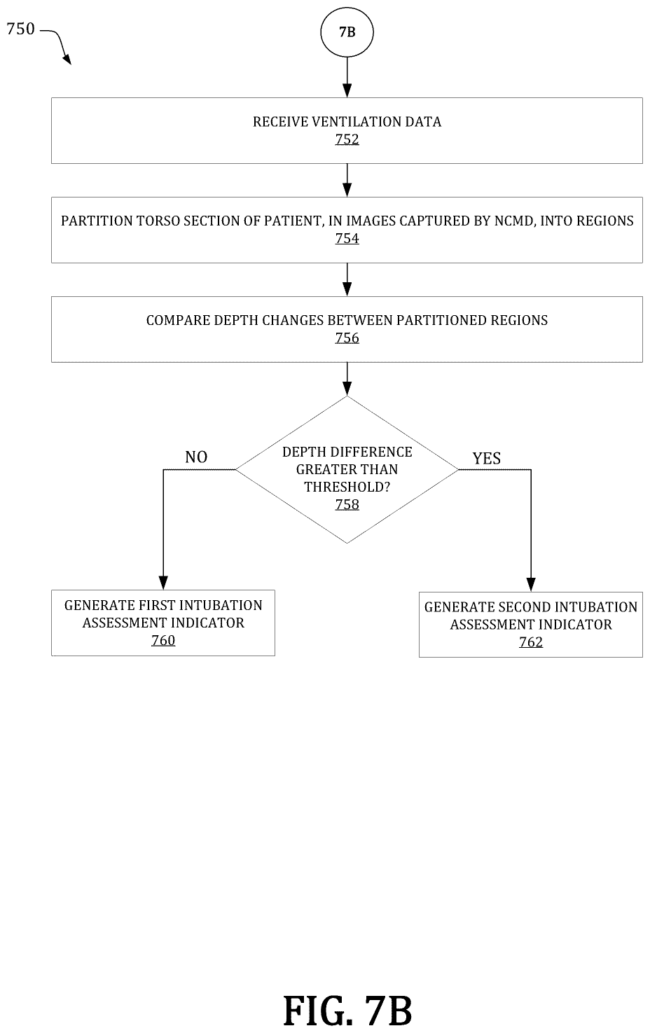

FIG. 7A depicts a method 700 for entering and exiting an intubation mode on an anesthesia machine. The operations of method 700 may be performed by the anesthesia machine. For instance, a memory of the anesthesia machine may store instructions that, when executed by one or more processors of the anesthesia machine, cause the anesthesia machine to perform the operations of method 700.

At operation 702, the anesthesia machine is initialized. The initialization may include the receipt of initial settings for the particular patient and/or other operations to prepare the anesthesia machine to provide medical gases and anesthetic to the patient. The initialization of the anesthesia machine may also include providing the medical gases to the patient via a mask prior to an intubation procedure.

At operation 704, the sensor data is received, processed, and displayed in a first configuration or mode, such as a normal operating mode (e.g., a non-intubation mode). The sensor data may be any sensor data that may be used to generate the patient parameter data discussed herein. For instance, the sensor data may include capnography data, electrocardiogram (ECG) data, SpO2 data, heart rate, blood pressure, temperature, depth of anesthesia level (e.g., bispectral (BIS) index data), nociception levels or data (e.g., Nociception Level (NOL)), and/or neuromuscular blockade data (e.g., Train-of-Four), among other types of data that may be displayed and/or used in the processes discussed herein. The patient parameter data may be displayed on one or more of the displays of the anesthesia machine, such as the MPM display.

At operation 706, image data is received from the video laryngoscope and/or the NCMD. For instance, the video feed from the video laryngoscope and/or the NCMD may be received by the anesthesia machine. Operation 706 may also include automatically pairing the video laryngoscope and/or the NCMD with the anesthesia machine. This pairing may be between an internal video laryngoscope pairing section of the anesthesia machine and/or with a removable module that has been plugged into the anesthesia machine or a component thereof. An example pairing process is discussed in U.S. Provisional Patent Application No. 63/505,275, titled Video Laryngoscope and Medical Device Wireless Video Transfer, which is incorporated herein by reference in its entirety.

At operation 708, an indication to enter an intubation mode is received. The indication may be the occurrence of an automatic trigger condition and/or a selection of an input element of the anesthesia machine, such as a button. For example, the anesthesia machine may receive a selection of a start intubation mode button of the anesthesia machine. In other examples, the sensor data and/or the video feed from the video laryngoscope may be analyzed to identify trigger criteria that, when satisfied, causes the anesthesia machine to enter the intubation mode.

As one example, changes in the capnography data may indicate that an intubation procedure has started and the intubation mode should be entered. For example, prior to the intubation procedure being performed, the patient may be ventilated (e.g., provided with medical gases) by the anesthesia machine via a mask or other patient interface other than an ETT. A capnography sensor positioned on the patient circuit or the patient interface detects the carbon dioxide concentration of the gases passing by the sensor. During an inhalation phase of a breath, the carbon dioxide value is relatively low as the patient is being delivered gases that are rich in oxygen and/or low in carbon dioxide (e.g., at or below concentrations of ambient room air). During an exhalation phase of a breath, the carbon dioxide exhaled by patient is detected by the capnography sensor. Accordingly, as long as the patient is breathing and connected to the patient circuit with the capnography sensor, a carbon dioxide signal is registered for each exhalation from the patient. This capnography data can be plotted over time as a capnogram, and an end tidal carbon dioxide value (EtCO2) value may be determined for each breath. Accordingly, when the mask is disconnected from the patient to begin the intubation procedure, the carbon dioxide signals associated with patient exhalations are no longer detected.

This absence of a capnography signal may be used as a trigger criteria for entering the intubation mode. For instance, detecting the absence of a capnography signal (above a threshold level) for a threshold duration may cause the anesthesia machine to automatically enter the intubation mode. As another example, the time from the last detected capnography signal (or the last detected EtCO2 value), may be referred to as the apneic time. Upon the apneic time reaching a threshold duration, the intubation mode may be initiated. The timer for the intubation mode may then be based on the time the intubation mode was initiated and/or the beginning of the apneic time, which may be more clinically relevant. In other examples, when the apneic time reaches a threshold duration, a notification is presented on the anesthesia machine that suggests that an intubation mode should be entered. The clinician may then confirm the start of the intubation mode by interacting with the notification or another button to activate the intubation mode. As discussed further below, the apneic timer ends when the capnography signal is again detected after the intubation procedure, which may also trigger an exit of the intubation mode.

Another automatic criterion for entering the intubation mode may be based on the video feed from the video laryngoscope. For example, when an anatomical feature such as a mouth, throat, or other feature is detected in the video feed, the intubation mode may be automatically entered. Such a detection may occur by an analysis of the video data at the video laryngoscope or at the anesthesia machine. Such a detection may also be used as one factor among multiple factors of the triggering criteria for entering the intubation mode.

Additional and/or alternative automatic criteria for entering the intubation mode may also be based on data from the NCMD. For example, as discussed above, depth data captured by the NCMD may be used to determine depth changes in the patient's torso, such as the chest of the patient. Accordingly, respiratory parameters such as breathing rate may be determined. When the chest of the patient ceases movement (or movement is below a threshold value), the breathing of the patient has ceased. Thus, a detection of a lack of movement of the chest based on the depth data from the NCMD may be used as an indicator to enter the intubation mode.

The images (e.g., video) captured by the NCMD may also be analyzed to identify indicia to enter the intubation mode. For instance, as discussed above, object recognition may be performed on the video captured by the NCMD (with or without the depth data) to identify objects within the images, such as medical objects (e.g., mask, ETT, video laryngoscope) and/or anatomical features of the patient (e.g., face, head, mouth). Based on the detected objects, the beginning of an intubation procedure may be detected. As an example, the removal of the mask from the patient may be used as an indicator to start the intubation mode. Detection of the removal of the mask may be performed by recognizing the mask object within the images and a face and/or mouth object of the patient. When the mask object is no longer overlapping with the face and/or mouth of the patient, the mask may be detected to have been removed. Intubation-mode indicia may also be detected when a video laryngoscope and/or an ETT are detected as being inserted into the mouth of the patient (e.g., when the video laryngoscope object and/or the ETT object overlap with a mouth or face object of the patient).

The indicia for entering an intubation mode may be assessed together, such as a weighted combination, in determining whether to begin the intubation mode. In other examples, a single indicia may be used in determining whether to begin the intubation mode.

At operation 710, the intubation mode timer is started upon entering the intubation mode at operation 708. In some examples, the intubation mode timer starts from a zero value corresponding to the same time that the intubation was entered, such as when a clinician selects a button to enter the intubation mode. In other examples, the intubation mode timer is aligned with, or based on, the apneic time based on the capnography signals. In such examples, the intubation timer represents the apneic time.

At operation 712, based on the anesthesia machine entering the intubation mode, a second display configuration is presented on one or more of the displays of the anesthesia machine. For example, an intubation-mode interface may be displayed instead of the first display configuration of the patient parameter data that was displayed in the non-intubation mode. The second display configuration, or intubation-mode interface, may include the features and segments described above, such as in the example displays shown in FIGS. 4A-4B. For instance, the video feed from the video laryngoscope (and/or the NCMD) may be displayed on a display of the anesthesia machine. In addition, the displayed patient parameters may be adjusted to display a subset of the patient parameters that are most relevant to the intubation procedure. In one example, the subset of patient parameters include ECG data and SpO2 data. In another example, the subset of patient parameters further includes capnography data. In still further examples, the subset of patient parameters further includes blood pressure (e.g., arterial pressure), depth of anesthesia level (e.g., bispectral (BIS) index data), nociception levels or data (e.g., Nociception Level (NOL)), and/or neuromuscular blockade data (e.g., Train-of-Four). Numbers representing the patient parameter data and/or the corresponding waveforms may be displayed as part of the intubation-mode interface.

At operation 714, an indication to exit the intubation mode is received. The indication may be based on a manual selection of a button of the anesthesia machine to end the intubation mode. In other examples, the indication to exit the intubation mode is generated automatically based on a set of trigger criteria being satisfied. For instance, the sensor data and/or video feed from the video laryngoscope may be analyzed to identify trigger criteria that causes the anesthesia machine to exit the intubation mode.

As one example, the capnography data may be analyzed and used to automatically exit the intubation mode. As discussed above, the presence of the carbon dioxide detected by the capnography sensor is based on exhalations from the patient. When the intubation procedure is completed, the patient circuit is connected to the ETT such that the medical gases flow into the patient from the anesthesia machine through the ETT. Similarly, exhaled gases flow through the ETT and pass the capnography sensor, where the carbon dioxide is detected. Accordingly, when the intubation procedure is completed and the patient exhales, carbon dioxide levels above a threshold are detected by the capnography sensor. Not only does the detection of such carbon dioxide indicate the end of the intubation procedure, but the presence of the carbon dioxide further indicates that the intubation procedure has been performed correctly. For example, during the intubation procedure, one potential mistake is to position the ETT in the esophagus rather than the trachea. When the ETT is mis-positioned in the esophagus, no carbon dioxide (or minimal carbon dioxide) flows through the ETT from the patient. As such, the lack of a capnography signal may also indicate an incorrect positioning the ETT. Thus, the intubation mode may be exited upon the detection of a capnography signal over a threshold for a threshold duration, such as a number of breaths (e.g., at least two breaths, 3-5 breaths).

As another example, the video feed from the video laryngoscope may be analyzed (either at the video laryngoscope or the anesthesia machine) to identify key events during the intubation procedure that indicate that the intubation has been completed. As one example, analysis of the video feed may include analyzing the individual image frames of the video feed to identify the presence of the ETT and the vocal cords in the video. The location of the tip of the ETT may similarly be identified and tracked over the frames in the video feed. The point in time where the tip of the ETT has passed through the vocal cords may be used as an indicator that the intubation procedure has been completed.

The object detection and tracking features of the present technology may be accomplished through the use computer vision and/or AI/ML models that process the frames of the video feeds. In one example, a trained convolutional neural network (CNN) may be used to process the image frames of the video feed. A CNN is a class of deep neural networks that are effective at analyzing visual imagery. CNNs are composed of layers that include convolutional layers, pooling layers, and fully connected layers. The convolutional layers apply various filters to the input to create feature maps, which highlight specific features in the image. In the initial layers, simple features like edges and colors may be detected. As the data progresses through the network, more complex features like textures and patterns are identified. Some methods generate potential bounding boxes in the image where objects might be located. For each bounding box, the CNN predicts the probability of each object class (e.g., ETT or vocal cords). The final output includes the class labels (e.g., vocal cords, ETT) and bounding box coordinates for each detected object in the image or video frame. Based on the overlap and relative positions of the detected bounding box for the ETT tip and bounding box for the vocal cords, an algorithmic determination may be made as to whether the ETT tip has passed through the vocal cords. In other examples, the combination of vocal cords with an inserted ETT may be its own class that is detectable by the ML model, and the detection of that class may indicate that the intubation procedure has been completed.

Other indicia that the intubation procedure may be complete include that the SpO2 value is increasing after a period of the decrease during the apneic period. Other patterns within the waveforms or trends of patient parameters may also be analyzed to determine indicia that the intubation procedure has been completed.

As yet another example, the data from the NCMD may also be analyzed and used as an indicator to automatically exit the induction mode. For instance, detection of chest motion based on the depth signals indicates that breathing has resumed (e.g., breaths are being delivered from the anesthesia machine and/or other ventilator). A detection that the video laryngoscope has been removed from the face or mouth of the patient may also be used as an indicator that the intubation procedure has been completed.

A combination of indicators may also be used as the criteria to exit the intubation mode. For example, the intubation mode may exit only when both the capnography signal is detected and the video confirmation of the ETT tube passing through the vocal cords is determined. The detection of the ETT passing through the vocal cords may occur prior to the detection of the capnography signal. Accordingly, in some examples, an indicator may be displayed in the intubation-mode interface that indicates a successful passage of the ETT into the trachea prior to the intubation mode being exited.

At operation 716, the intubation timer is stopped upon receiving the indication to exit the intubation mode. The total duration of the intubation procedure may then remain displayed (e.g., the total time on the intubation timer when the timer is stopped).

In some examples, method 700 may also proceed to method 750 depicted FIG. 7B. FIG. 7B depicts an example method 750 for assessing an intubation based on depth data captured by an NCMD. The assessment of the intubation may be performed to detect whether the intubation procedure was performed as intended (e.g., non-esophageal intubation, proper main-stem intubation).

At operation 752, ventilation data is received or accessed. The ventilation data indicates the current ventilation parameters of the ventilation being provided to the patient by the anesthesia machine (or other ventilator). For instance, the ventilation data may include real-time data about the flow of gases into and/or out of the patient (e.g., inhalation and/or exhalation data). Such data may include flow rates, pressures, volumes, etc. The ventilation data may be generated by the respective sensors of the anesthesia machine (or other ventilator). Other types of ventilation data may include respiratory rate or set tidal volume, among others. The received ventilation data may then be used in assessing changes in depths of the patient's torso (e.g., chest). For instance, the real-time ventilation data may indicate that a current breath is being delivered to the patient (e.g., breathing gases are being delivered). Thus, there is an expectation that the chest region of the patient should expand due to the lungs filling with gas. This expectation may be used in assessing the depth measurements from the NCMD.

At operation 754, a torso section of the patient is identified in the video data from the NCMD and virtually partitioned into different regions. The particular regions for which the torso is partitioned may depend on the type of intubation that is being performed. For instance, potentially for all intubations, the torso may be portioned into a chest region and an abdomen (or stomach) region. Similarly, the chest region may be portioned into a left chest region (corresponding to a left lung) and a right chest region (corresponding to a right lung). For intended main-stem intubations, the left chest region and the right chest region may be further portioned into upper and lower subregions. For example, the partitioning may result in an upper left chest subregion, a lower left chest subregion, an upper right chest subregion, and a lower right chest subregion.

At operation 756, depth changes between the partitioned regions are compared. For example, the depth changes for each region may be determined over a period of time. The period of time may correspond to an inhalation phase of a delivered breath, as indicated by the ventilation data received in operation 752.

Depending on the particular assessment desired and/or the intended type of intubation, different regions may be compared to one another. For example, a depth change of the abdomen region may be compared to a change in depth of the chest region to attempt to identify an improper esophageal intubation. The depth changes of the left chest region and the right chest region may be compared to identify asymmetrical inflation of the left and right lungs. The depth changes of the lower left chest subregion may be compared with the depth changes of the upper left chest subregion to identify asymmetrical inflation of the left lung.

At operation 758, a determination is made as to whether the compared depth difference is greater than a set threshold amount. The set threshold amount may be based on the particular comparison being performed. For instance, a first threshold value may be used for a comparison of the abdomen and the chest region, and a different, second threshold value may be used for a comparison of the left chest region and the right chest region. Yet another different, third threshold value may be used for the comparison of the upper and lower chest subregions. The threshold values may be absolute values (e.g., differences in distances, volumes, etc.) and/or percentage differences between the compared depth changes. The threshold values may also be based on characteristics of the particular patient. For instance, absolute depth changes for a smaller patient (e.g., neonatal patient) are different than for a larger patient (e.g., adult patient). As such, the threshold values may be based on patient type (e.g., pediatric versus adult), sex, height, weight, and/or other patient characteristics. The threshold values may also be adjusted based on the ventilation data in some examples. For instance, the expected depth changes may differ between larger delivered tidal volumes versus smaller delivered tidal volumes.

If the depth difference is not greater than the respective threshold, the method 750 flows to operation 760 where a first intubation assessment indicator is generated. If the depth difference is greater than the respective threshold, the method 750 flow to operation 762 where a different, second intubation assessment indicator is generated.

The first and second intubation assessment indicators may indicate the presence (or lack of) asymmetry. For instance, the first intubation assessment indicator indicates that a symmetry has been detected. The second intubation assessment indicator indicates that an asymmetry has been detected.

The indicators may also provide additional information based on the type of intubation that was intended to be performed. For example, inflation of the abdomen, whether asymmetric or symmetric with the chest may cause an indication of a potential esophageal intubation to be generated. For an intended tracheal intubation (e.g., non-main-stem), substantially symmetry between the left lung and the right lung is the desired outcome. Accordingly, in such examples, the first intubation assessment indicator may indicate a proper intubation procedure and the second intubation indicator may indicate an improper intubation procedure. In contrast, for an intended main-stem intubation, asymmetry between the left lung and the right lung is the intended result. As such, the second intubation indicator may indicate a proper main-stem intubation procedure, and the first intubation indicator may indicate an improper main-stem intubation procedure. For a main-stem intubation, symmetry between the upper and lower lung subregions however may be desired. Thus, the indicators may be different where the comparison is between the upper and lower lung subregions.

Returning to method 700 in FIG. 7A, at operation 718, the data that was recorded during the intubation mode is provided to the patient terminal for inclusion in the EMR for the patient. For instance, the video data from the video laryngoscope and/or the NCMD that was received during the intubation mode and the patient parameters that were recorded during the intubation mode are transmitted to the patient terminal for inclusion in the EMR and/or for use in filling in the EMR by the clinician.

At operation 720, upon exiting the intubation mode, the display is reverted to the first display configuration according to the non-intubation mode. For example, where the MPM display is updated to show an intubation-mode interface, the intubation-mode interface is removed and the MPM display is reverted to showing the patient parameters associated with the non-intubation mode. This adjustment may include removing the video feed from the video laryngoscope and/or NCMD from display.

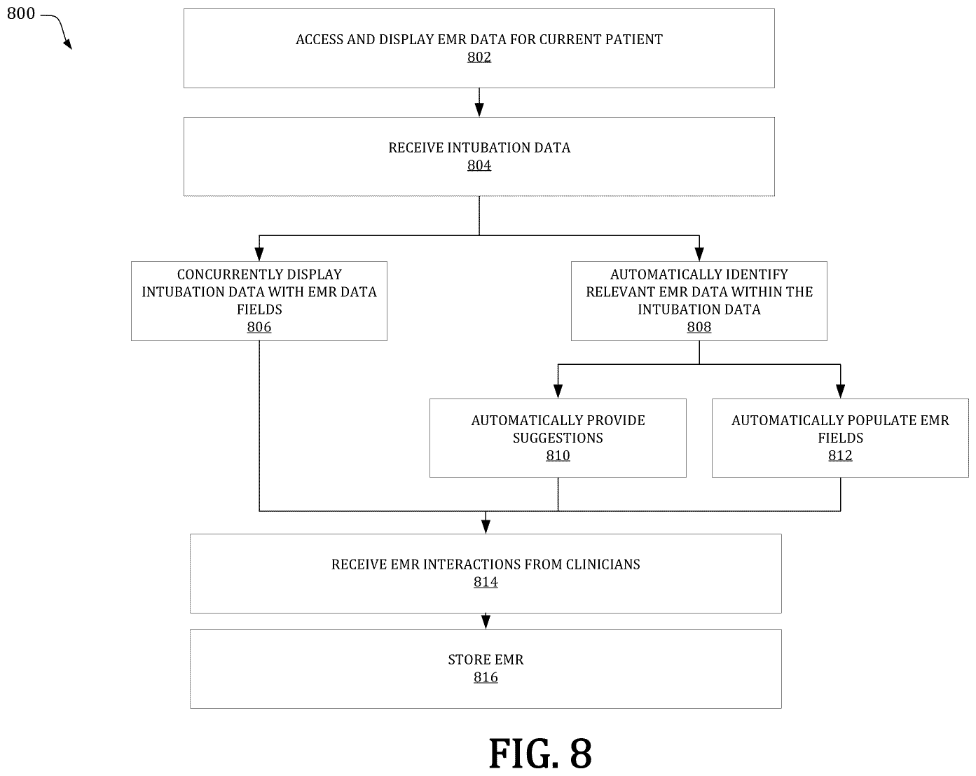

FIG. 8 depicts a method 800 for populating an electronic medical record with data associated with intubation mode. The operations of method 800 may be performed by the patient terminal and/or other components of the anesthesia machine.

At operation 802, EMR data for the current patient is accessed and displayed. Accessing the EMR data may include accessing a remote database storing the EMR data for the patient, such as through a web portal or specialized application operating on the patient terminal. For instance, a clinician may need to input particular credentials to gain access to the database and provide identifying information about the patient to retrieve the EMR for the patient. The access may be performed over a network connection, such as a hospital network connection. The connection may provide access to databases or servers that are located on premises of the hospital and/or cloud-based servers accessible via the Internet.

With the patient EMR accessed, the intubation data associated with the intubation mode is received in operation 804. For instance, the video data from the video laryngoscope and/or NCMD received during the intubation mode and the patient parameter data recorded during the intubation mode may be received as the intubation data. In some examples, the video data may include still screenshots from the video feed(s). For instance, during the intubation procedure, the clinician may capture still images (e.g., screenshots), which may be included in the video data. In other examples, the video data may also include objects that were identified within the video feed(s) (e.g., ETT, mask, vocal cords) and time stamps at which such objects were identified. For instance, upon objects and/or events being identified in the video feed, screenshots of those images or shortened portions of the video surrounding the events (e.g., 5-10 second clips) may be identified or included in the video data. The video data may also include additional data extracted from the video feed(s). For instance, the computer vision technology discussed herein may be able to further classify the particular ETT based on its size. As a result, the size of the ETT can automatically be identified from the video data. The type and/or size of the laryngoscope blade used on the video laryngoscope may also be able to be automatically identified from the video data. In addition, the video data may be analyzed to generate a difficulty score for the intubation procedure. The video data may also be analyzed to identify any anatomical anomalies, such as lesions, discolorations, or other abnormalities. For instance, the coloring of the anatomy may be analyzed to determine departures from expected coloring, such as green vocal cords. Such detected anomalies and/or abnormalities may be included in the intubation data and used to populate the EMR.

The other intubation data may also include the duration of the intubation mode (e.g., the timer value duration). The number of intubation attempts may also be indicated in the intubation data. The number of attempts may be determined by the number of times the second attempt button was selected. In other examples, the video data from the video laryngoscope may be analyzed to identify the number of attempts that were performed.

With respect to the patient parameters included in the intubation data, all the available patient parameter data recorded during the intubation mode may be included in the intubation data. In other examples, only the patient parameter data displayed in the intubation-mode interface during the intubation mode is included in the intubation data. In some examples, the patient parameter data is further analyzed to extract minimums, maximums, or other anomalies, such as a lowest SpO2 level and/or a blood pressure spike.

By receiving the intubation data while the patient-specific EMR is accessed and open on the patient terminal, the intubation data is automatically implicitly associated with the patient undergoing the intubation procedure without having to tag the intubation data itself. For instance, by incorporating the intubation data into the patient's EMR during the surgical procedure, the intubation data itself does not need to be stored separately and identified with the patient. Such an enhancement reduces potential privacy risks and significantly reduces storage and data management requirements for such personal health data.

The intubation data may be provided through an AIMS interface or similar interface. The intubation may be received from the anesthesia machine, such as from the MPM section of the anesthesia machine. In other examples where the video laryngoscope wireless module is plugged into the patient terminal directly, the video laryngoscope video data may be received via the wireless module rather than from the anesthesia machine or MPM section thereof.

Once the intubation data is received, operation 806 and/or operation 808 may be performed. In operation 806, the intubation data is displayed with the fillable fields of the EMR for the patient. In some examples, the intubation data is displayed concurrently with the fillable fields on the patient terminal. Accordingly, the clinician can more quickly and accurately populate the fillable fields. The display of the intubation field may include video player with the video data from the video laryngoscope and/or NCMD. The clinician may be able to navigate through the video to identify particular events or features that are desired to be reviewed by the clinician. Captured still screenshots may also be displayed along with other data captured by the classifications of the objects within the video feed(s).

At operation 808, data within the intubation data that is relevant to the fillable fields of the EMR data is automatically identified. For instance, for a topic of a fillable field, a search query may be executed over the intubation data may be performed to identify the data that is relevant to the particular field. If the computer vision techniques to extract data from the video feed(s) have not been previously performed, such techniques may be performed at operation 808 and targeted to extract data specific to the particular fields of the EMR for the patient.

At operation 810, based on the relevant data identified in operation 808, suggestions for one or more of the fillable fields of the EMR are presented. For example, suggested entries based on the intubation data may be displayed adjacent to one or more fillable fields for which relevant data was identified. A clinician may then interact with the suggested data to cause the data to be populated in the respective field. At operation 812, the fillable fields for which relevant intubation data was identified are automatically populated with identified relevant data. Such automatic population or suggestions in operations 810 and/or 812 greatly reduces the number of inputs that the patient terminal needs to process from the clinician and also reduces the total time required from the clinician to complete the EMR for the patient before being able to complete additional medical procedures.

At operation 814, interactions with the patient terminal are received. The interactions may be to simply accept the automatically populated data in the EMR. In other examples, the interactions may edit or adjust the data within the EMR. The interactions may also further augment the EMR data, such as by adding notes or other narratives to the EMR for the patient. The clinician may then accept and submit the EMR for storage.

At operation 816, once the clinician has edited and/or accepted the entries within the EMR, the updated EMR is stored. The EMR may be stored locally on the patient terminal and/or uploaded to a database on a hospital server or cloud-based server for storage and later remote access. The EMR may include not only the intubation data that was incorporated into the fillable fields of the EMR, but the EMR may also include the intubation data itself, such as a copy of the video file(s) from the video laryngoscope and/or the NCMD.

Once the EMR is saved and stored, the intubation data stored in other location may be deleted. For example, once the intubation data has been stored in the EMR (or at least the relevant portions thereof), the intubation data may be deleted from the other memory of the anesthesia machine, such as the MPM section memory or primary memory of the anesthesia machine. A signal may also be transmitted back to the video laryngoscope to indicate that the corresponding video file can be deleted from the memory of the video laryngoscope. The video laryngoscope may then delete the corresponding video file from its memory.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. An anesthesia machine comprising:
a display;
at least one processor; and
memory storing instructions that, when executed by the at least one processor, cause the anesthesia machine to perform operations comprising:
receive sensor data from one or more connected sensors;
display, on the display, patient parameters based on the received sensor data, wherein the patient parameters are displayed in a first display configuration;
receive video data from a video laryngoscope;
receive an indication to enter an intubation mode;
in response to the indication, display, on the display, the video data and the patient parameters in a second display configuration;
receive an indication to exit the intubation mode; and
in response to the indication to exit the intubation mode, remove the video data from the display and display the patient parameters in the first display configuration.

2. The anesthesia machine of claim 1, wherein the operations further comprise:
in response to the indication to enter the intubation mode, starting an intubation timer; and
in response to the indication to exit the intubation mode, stopping the intubation timer.

3. The anesthesia machine of claim 2, wherein the operations further comprise concurrently displaying the intubation timer with the video data and the patient parameters in the second display configuration.

4. The anesthesia machine of claim 1, wherein the operations further comprise, in response to the indication to exit the intubation mode, transmitting, to a patient terminal for inclusion in an electronic medical record, the video data receiving during the intubation mode and the patient parameters generated during the intubation mode.

5. The anesthesia machine of claim 1, wherein the indication to enter the intubation mode is based on an absence of a capnography signal from a capnography sensor.

6. The anesthesia machine of claim 5, wherein the indication to exit the intubation mode is based on detecting a capnography signal from the capnography sensor.

7. The anesthesia machine of claim 1, wherein the indication to exit the intubation mode is based on detecting, from the video data, that a tip of an endotracheal tube has passed through vocal cords.

8. The anesthesia machine of claim 1, wherein the indication to enter the intubation mode is based on receiving a selection of a button of the anesthesia machine.

9. A computer-implemented method for incorporating intubation data into an electronic medical record of a patient, the method comprising:
accessing the electronic medical record of the patient;
receiving, from an anesthesia machine, intubation data generated during an intubation procedure of a patient, wherein the intubation data includes patient parameters generated during the intubation procedure and video data, from a video laryngoscope, captured during the intubation procedure;
concurrently displaying at least a portion of the intubation data with fillable fields of the electronic medical record;
receiving at least one interaction with the electronic medical record during the concurrent display; and
storing the electronic medical record based on the at least one interaction.

10. The computer-implemented method of claim 9, wherein concurrently displaying at least the portion of the intubation data includes displaying a video player with the video data.

11. The computer-implemented method of claim 9, further comprising executing a query over the intubation data to identify intubation data relevant to at least one of the fillable fields of the electronic medical record.

12. The computer-implemented method of claim 11, further comprising displaying one or more suggested entries to one or more of the fillable fields based on the identified relevant intubation data.

13. The computer-implemented method of claim 11, further comprising automatically filling one or more of the fillable fields with the identified relevant intubation data.

14. The computer-implemented method of claim 9, wherein the intubation data includes data extracted from the video data including at least one of an endotracheal tube size and a difficulty score for the intubation procedure.

15. The computer-implemented method of claim 9, wherein storing the electronic medical record includes storing the video data with the electronic medical record.

16. An anesthesia machine comprising:
a display;
at least one processor; and
memory storing instructions that, when executed by the at least one processor, cause the anesthesia machine to perform operations comprising:
receive sensor data from one or more connected sensors;
receive first video data from a video laryngoscope;
receive second video data from a non-contact monitoring device (NCMD);
based on a first indicator generated from at least one of the sensor data, the first video data, or the second video data, automatically entering an intubation mode, wherein entering the intubation mode includes displaying at least one of the first video data and the second video data on the display; and
based on a second indicator generated from at least one of the sensor data, the first video data, or the second video data, automatically exiting the intubation mode.

17. The anesthesia machine of claim 16, wherein the first indicator is based on a detection, from the second video data of a mask removal.

18. The anesthesia machine of claim 17, wherein the second indicator is based on a capnography signal in the sensor data.

19. The anesthesia machine of claim 16, wherein entering the intubation mode causes an intubation timer to start, and exiting the intubation mode causes the intubation timer to stop.

20. The anesthesia machine of claim 16, wherein the second video data further includes depth data captured by the NCMD.
